(19) United States
(12) Patent Application Publication
(10) Pub. No.: US 2005/0282237 A1
(43) Pub. Date: Dec. 22, 2005

(54) Immunological analysis carrier and an immunological analysis method using the same

(76) Inventor: Yoshio Ishimori, Kawasaki-shi (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 11/145,931

(22) Filed: Jun. 7, 2005

(30) Foreign Application Priority Data
Jun. 8, 2004 (JP) 2004-170056

(51) Int. Cl. G01N 33/53; G01N 33/537; G01N 33/543; C12M 1/34

(52) U.S. Cl. 435/7.92; 435/287.2

(57) ABSTRACT

The present invention provides an immunological analysis carrier comprising at least one of an antibody against a target substance, an agent capable of specifically binding a target substance, a part of an antibody against a target substance and a part of an agent capable of specifically binding a target substance, the at least one of the antibody, the agent, the part of the antibody and the part of the agent being immobilized on the carrier's surface.

### CROSS-REFERENCE TO RELATED APPLICATIONS

This application is based upon and claims the benefit of priority from prior Japanese Patent Application No. 2004-170056, filed Jun. 8, 2004, the entire contents of which are incorporated herein by reference.

### BACKGROUND OF THE INVENTION

1. Field of the Invention

The present invention relates to an immunological analysis carrier and an immunological analysis method using the same. More particularly, the present invention relates to an immunological analysis reagent used for specific detection of and quantitative or qualitative analysis of a selected substance in a sample and an immunological assay method using the same.

2. Description of the Related Art

Radioimmunoassay (hereinafter referred to as “RIA”) has been used generally for quantitative analysis of target substances such as trace amounts of antigens or antibodies in a sample. However, RIA has a defect associated with the use of radioactive elements, i.e. installation of a dedicated instrument and its operation by a qualified operator are needed. There is also a problem with disposal of wastes. Other known analysis method is, for example, electrophoresis. However, electrophoresis requires a lot of time for measurement, and it cannot be used for analyzing target substances small in amount due to low sensitivity.

In Japanese Patent Publication 60-117159, we have disclosed an immunological analysis reagent comprising a liposome (a microcapsule comprised of lipid membranes) encapsulating a hydrophilic marker substance therein and having a hydrophilic antibody or antigen covalently immobilized on its surface. This reagent is used for immunological analysis as follows. This immunological analysis reagent is added to a sample containing antigens or antibodies. Subsequent addition of complements disrupts the liposome through an antigen-antibody reaction and concomitant action of the complements, causing discharge of the encapsulated marker substance (e.g. fluorescent compound). The known correlation between the amount of discharged marker substances and the amount of the target substances in the sample allows for quantitative determination of the target substances by quantitatively determining the discharged marker substances using a particular analysis method (e.g. fluorescent analysis). This reagent will simplify immunological analysis by eliminating problems associated with RIA.

However, it was found that analysis of samples containing serum or protein using the immunological analysis reagent involved non-specific reactions besides an antigen-antibody reaction and these non-specific reactions could disrupt liposomes. These reactions are suggested to be caused by a reaction between proteins/race chemicals/complements in a sample and liposomes. For this reason, the analysis has been performed after diluting a sample containing serum or protein.

For example, when α-fetoprotein (AFP) in human serum is to be analyzed using an immunological analysis reagent which comprises liposomes having anti-human α-fetoprotein antibodies (hereinafter referred to as “anti-human AFP antibody”) immobilized thereon, human serum is diluted ×100 to eliminate effects from the non-specific reactions. The serum AFP concentration of a normal subject is below 10 ng/mL. Therefore, after ×100 dilution of serum from a normal subject, AFP in a concentration below 0.1 ng/mL should be measured by, for example, fluorescent analysis. This demands highly sensitive analysis. Also, due to a large fluorescent detection device needed for precise fluorescent analysis, whole analysis instruments have to be large and costly.

### BRIEF SUMMARY OF THE INVENTION

This invention was made to solve these problems and intended to provide an immunological analysis reagent that allows precise and simple analysis as well as an immunological analysis method using the same.

According to embodiments of the present invention, it is provided an immunological analysis carrier comprising: An immunological analysis carrier comprising: at least one of an antibody against a target substance, an agent capable of specifically binding a target substance, a part of an antibody against a target substance and a part of an agent capable of specifically binding a target substance, the at least one of the antibody, the agent, the part of the antibody and the part of the agent being immobilized on the carrier's surface; and a redox enzyme capable of generating an electrochemically active substance carried on the carrier's surface or encapsulated in the carrier.

The immunological analysis carrier and the immunological analysis method in accordance with the present invention allows for sensitive, yet inexpensive and compact immunological analysis.

### BRIEF DESCRIPTION OF THE SEVERAL VIEWS OF THE DRAWING

FIG. 1 is a principle diagram of an immunological analysis method using an immunological analysis carrier (microcapsule reagent) according to embodiments of the invention.

FIG. 2 shows a result of a measurement using the immunological analysis carrier (microcapsule reagent) of Example 1. The relative value of current and relative fluorescent intensity are shown when GOD and carboxyfluorescein were encapsulated, respectively.

### DETAILED DESCRIPTION OF THE INVENTION

In one embodiment of the invention, the immunological analysis carrier can be made of any materials. For example, a carrier made up of lipid molecules (liposome reagent) can be applied. The immunological analysis carrier of the invention is preferably in the form of a microcapsule. In this case, at least either of phospholipids or glycolipids can be used as a major constituent of a lipid composition. In some cases, other lipids such as cholesterol will optionally be added to stabilize a membrane. The phospholipids and glycolipids that can be used for the invention include, but not limited to, dipalmitoylphosphatidylcholine (DPPC), dipalmitoylphosphatidylethanolamine (DPPE), dioleoylphosphatidylethanolamine, dimyristoylphosphatidylethanolamine, distearoylphosphatidylethanolamine. Carbon chains of fatty acids in these phospholipids and glycolipids have preferably 12-18 carbon atoms, more preferably even number of carbon atoms. Commercially available liposome reagents can for example serve as these lipids. It is also preferable to previously determine and select lipids having optimal types and composition ratios in terms of a test substance, measurement sensitivity and stability of a liposome reagent. The liposome reagent used as a material for a microcapsule can easily be disrupted by giving osmotic shock or ultrasonic stimulation and the like to discharge enzyme molecules encapsulated therein.

Macromolecule compounds with general micellar structures can also be used as the immunological analysis carrier in the form of a microcapsule used for the invention, in this instance, the microcapsule can be disrupted by chemical stimulation such as pH change.

In addition, porous carriers can also be applied as the immunological analysis carrier used for the invention. An example of such porous carriers includes Sepharose CL (Amersham). When such porous carriers are used, the surface of the porous carriers can directly carry the enzyme.

The immunological analysis carrier of the invention has an antibody against a target substance or an agent capable of binding the target substance or a part thereof (hereinafter collectively referred to as “antibody and the like against a target substance”) immobilized on its surface. In the context of the present immunological analysis carrier, an antibody against a target substance or an agent capable of specifically binding the target substance (e.g. receptor) can be any protein capable of binding a target substance including IgG, IgE, IgD, IgA and IgM, or other organic molecule.

When an antibody is immobilized on the immunological analysis carrier, it is preferable to use polyclonal antibodies as opposed to monoclonal antibodies from the aspect of increasing sensitivity. In some cases, the antibody can be F(ab′)₂ antibody, which can be produced by removing Fc portions from an antibody with proteolytic enzymes like pepsin, Fab′, which can be produced by further reducing F(ab′)₂.

Now, we illustrate the present immunological analysis carrier and its manufacturing method in more detail, taking as an example where the liposome reagent is used as a material for the immunological analysis carrier in the form of a microcapsule.

The immunological analysis carrier according to the present invention has an antibody against a target substance or an agent capable of specifically binding the target substance or a part thereof immobilized on its surface. The target substance has no limitation. Thus, the immunological analysis carrier of the present invention allows detection of such target substances as macromolecules like general proteins and nucleic acids as well as micromolecular organic compounds like drugs such as narcotics and powders. However, the target substance must have more than one antigen determinants, because the target substance is detected by so-called sandwich method in the present immunological analysis method.

For example, functional groups such as halogenated acetyl groups can be use to immobilize antibodies and the like (e.g. antibodies) against the target substance onto a material for the immunological analysis carrier. In this case, the following group is introduced into phospholipids and glycolipids.
—CO(CH₂)ₘNHCOCH₂X group

Wherein m signifies a spacer linking the lipid molecules and the functional group portions and should be selected to have appropriate length within 0-12; X signifies either elements among Cl, Br, or I and can be selected as appropriate.

The spacer is introduced to reduce steric hindrance provided by an immobilizing carrier that may occur during a reaction between the target substance and an agent capable of binding the target substance. The functional group can be introduced using, for example, the following reaction.

In the instance where lipids including a spacer and a functional group (m is not 0) as described above, ω-amino acids such as 3-amino propionic acid (NH₂(CH₂)₂COOH) or 5-amino valeric acid (NH₂(CH₂)₄COOH) are protected at their amino groups, then reacted with amino group-containing lipids (e.g. DPPE) in the presence of triethyl amine together with N-hydroxysuccinimide (HSI) and N,N′-di-cyclohexylecarbodiimide (DCCD). The protecting groups are then removed with e.g. hydrochloric acid. Alternatively, ω-amino acids such as 3-amino propionic acid or 5-amino valeric acid can be protected at their amino groups to be followed by a reaction with HSI and DCCD in the presence of TEA for synthesizing succinimide ester. The resultant succinimide ester will then be reacted with the amino group-containing lipids followed by removal of the protecting groups.

Halogenated acetic acid is then reacted with the lipid attached with a spacer in the presence of TEA together with HSI/DCCD. Alternatively, ω-amino acids such as 3-amino propionic acid or 5-amino valeric acid can be protected by esterification at their carboxy groups, followed by attaching the halogenated acetic acid thereto. After deprotection of the protecting groups, this will be reacted with amino group-containing lipids in the presence of HSI/DCCD and TEA. For purification of lipids synthesized as mentioned above, thin layer chromatography for fractionation can conveniently be used.

Next, we will explain how to produce the liposome reagent. Phospholipids and/or glycolipids containing —CO(CH₂)ₘNHCOCH₂X groups produced as described above and aliphatic amines and optionally cholesterol and other lipids are added in a flask, then solved and mixed after adding solvents and dried by aspirating the solvents. This may form uniform lipid thin layers on a wall of the flask.

Subsequently, an appropriate concentration of aqueous solution of a redox enzyme will be added to the flask. Heating to an appropriate temperature followed by vigorous shaking with the flask sealed will give a suspension of multi-layer liposome. The redox enzyme to be encapsulated can be any redox enzyme known to those skilled in the art, and as an example, glucose oxidase can be used. Small unimembrane liposomes, which can be prepared by additional sonication of the suspension of the multi-layer liposomes will enhance a reaction amplifying effect. While the number of the redox enzyme encapsulated in this procedure will depend on its molecular weight, particle size and preparation method of the liposome, it will be approximately 100-100,000 per one liposome. The redox enzyme to be encapsulated is a macromolecule compound with a molecular weight of 10,000 or more. Non-redox enzyme generating an electrochemically active substance through reaction with its substrate can also be used.

The antibody and the like against a target substance will be provided with a free SH group using enzymatic treatment with e.g. pepsin or reductive treatment. Otherwise, a SH group can be introduced by a reaction with a bifunctional reagent such as SPDP (Pharmacia) followed by reduction. In addition, the antibody and the like against a target substance can be immobilized to liposomes via—CO(CH₂)ₘNHCOCH₂—bonding (wherein m is e.g. 2 or 4) by gently reacting the liposome suspension and the antibody and the like against a target substance in an appropriate buffer.

As illustrated below, the present immunological analysis carrier can be used in conjunction with separating particles. Magnetic microparticles can be applied as the separating particles and the antibody and the like against a target substance is immobilized on its surface. For the immobilization, covalent bonding and avidin-biotin reaction can, for example, be used in a similar way as the immunological analysis carrier above.

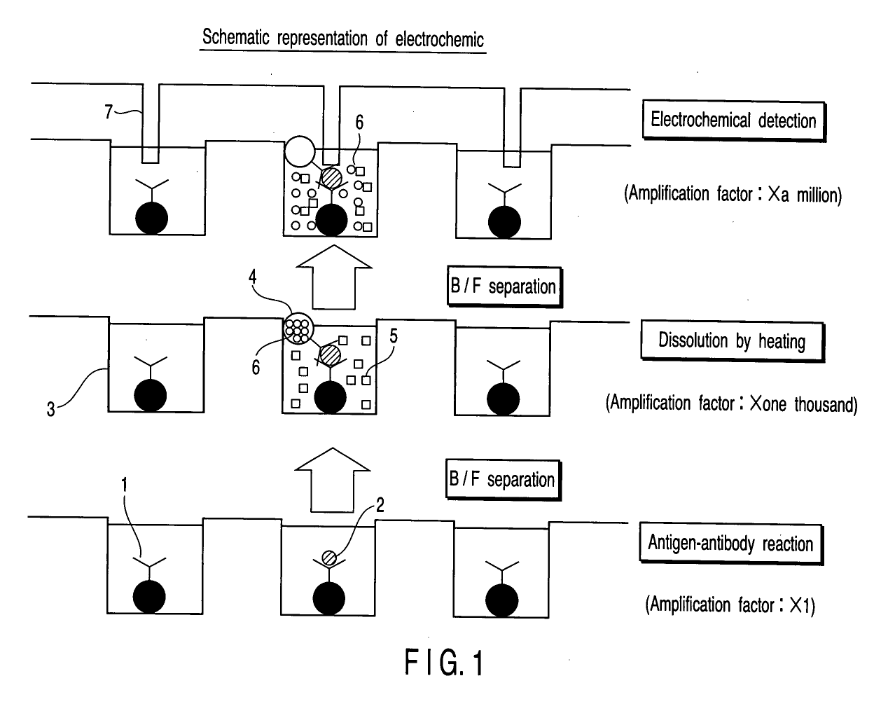

The present immunological analysis carrier above can be used as follows. In this instance, an antigen (target substance) is determined using the immunological analysis carrier having an antibody on its surface, and the separating particle. As mentioned above, the separating particle is a particle on which an antibody and the like against a target substance is previously immobilized (FIG. 1; see reference code 1). The antibody and the like against a target substance to be immobilized has preferably an antigen determinant different from that carried by the antibody and the like against a target substance bound on the present immunological analysis carrier. Although we mention an example where a magnetic microparticle is used as a separating particle, any particle can be used provided that it binds to the target substance and can be subjected to B/F separation.

First, separating magnetic particles are added to a sample containing a target substance under a constant temperature and subjected to reaction for a fixed period time. An appropriate amount of the immunological analysis carrier immobilized with an antibody against a target molecule is then added to get it bound to the target substance. Complexes consisting of the separating particles, the target substance and the immunological analysis carrier are separated from solution. The complexes consisting of the separating magnetic particles, the target substance and the immunological analysis carrier can be collected by way of binding of the separating magnetic particles to a magnet. These complexes are extensively washed with washing solution to wash off any unreacted immunological analysis carrier (liposome reagent). Microcapsules are then disrupted, if the immunological analysis carrier is in the form of a microcapsule encapsulating a redox enzyme. The microcapsules can be disrupted, for example, by adding an approximate amount of pure water. Substrate to the redox enzyme discharged by disruption is further added (FIG. 1, the middle panel). Substrate can be added without disruption procedure, if the immunological analysis carrier carries the redox enzyme on its surface. As a substrate, substrate to the redox enzyme encapsulated in or carried on the immunological analysis carrier is used, for example, glucose (in PBS solution) will be used when glucose oxidase is used as the redox enzyme. The substrate can be added in a concentration appropriate for the encapsulated redox enzyme, for example, 1% of glucose will be added when glucose oxidase is used.

Finally, the reaction between the redox enzyme and the substrate is detected. For example, electrochemical reaction associated with redox reaction can be detected by inserting a working electrode into reaction medium (FIG. 1; the lower panel). Electrochemical reaction can be detected by calculating the relative value of current as illustrated in the following examples.

For actual quantitative analysis, a standard curve will be previously plotted using known concentration of a target substance, and the magnitude of an electrical signal will then be measured which is generated by a reaction under the same condition with a sample containing unknown concentration of a target substance, and concentration of the target substance can then be quantitated based on the standard curve. The use of the present immunological analysis carrier allows highly-sensitive and qualitative detection of the presence of a target substance by simply mixing the immunological analysis carrier with a target substance for s sufficient period of time (appropriate period should be established prior to mixing, because the period will be varied depending on types of the target substance and the properties of the immunological analysis reagent) and measuring the magnitude of an electrical signal.

Conditions such as time and temperature needed for reaction between the immunological analysis carrier (e.g. microcapsulated liposome reagent) and a target substance-containing sample can be varied with types of the target substance, the properties of microcapsule, types of enzyme molecules, as well as with the types, amount and purity of an agent capable of specifically binding a target substance chemically bonded to the immunological analysis carrier or part thereof. Therefore, it is desirable, when plotting the standard curve, to make a preliminary determination each time using a sample containing a specified concentration of a target substance in order to establish the optimal reaction time and temperature.

Target substances which can be quantitated using the present immunological analysis carrier cover a diverse range of substances, and among these are included proteins including tumor markers in biological fluid such as serum (e.g. AFP, BFP, CEA, POA) and immunoglobulins (antibodies such as IgG, IgE, IgD and IgM), hormone (e.g. insulin, T₃), and micromolecular compounds such as drugs including narcotics and powder.

The present immunological analysis carrier can be provided in the form of a kit, together with materials necessary for detecting a target substance, for example, together with separating particles, enzyme substrates, and/or other suitable reagents and the like.

### EXAMPLES

In one embodiment of an immunological analysis carrier according to the present invention, an experiment was performed, by way of an example, using a measurement system for AFP (a-fetoprotein; hepatic tumor marker) by means of a liposome reagent. FIG. 1 is a schematic representation of this analysis system. Among the reagents used in these examples, dipalmitoylphosphatidylcholine (DPPC), dipalmitoylphosphatidylethanolamine (DPPE), and cholesterol were purchased from Sigma, and for other reagents commercially available reagents (special grade) were used without further purification. Water used was all ion-exchanged water.

### Example 1

### Preparation of Human IgG-Immobilized Liposome (Containing Lipid Including a Spacer (m=4) and a Functional Group, and Stearylamine)

1. Synthesis of NH₂-C₅-DPPE

(a) Synthesis of Boc-5-Amino Valeric Acid

30 mL (approx. 20 mmol) of triethylamine (TEA) and 10 mL of water were added to 1.17 g (10 mmol) of 5-amino valeric acid (Aldrich) to solve it. 2.7 g (11 mmol) of Boc-ON (Peptide Laboratory), which serves as a protecting group for amino group, in 10 mL of dioxane was added to it, and stirred for three hours at room temperature. After completion of the reaction, reaction solution was concentrated by a rotary evaporator and extracted and purified sequentially with ethyl acetate, 5% aqueous sodium bicarbonate, and 5% aqueous citric acid. Finally, the product was dehydrated by anhydrous sodium sulfate, and crystallization under low temperature gave Boc-5-amino valeric acid. The yield was about 70%.

(b) Synthesis of Boc-5-Amino Valeric Acid Succinimide ester

0.23 g (1 mmol) of the Boc-5-amino valeric acid was solved in 20 mL of chloroform and 0.13 g (1.1 mmol) of N-hydroxysuccinimide (HSI; Peptide Laboratory) and 0.25 g (1.2 mmol) of dicyclo-hexylcarbodiimide (DCCD; Peptide Laboratory) were added, and then stirred at room temperature for three hours. After completion of the reaction, solvents were removed by a rotary evaporator, and solved by adding 30 mL of ethyl acetate to the resultant product, and the precipitate was removed by filtration. Again, solvents were removed, and the product was solved in 5 mL of chloroform to use for the following reaction as Boc-5-amino valeric acid succinimide ester solution (assumed as approx. 0.2 mmol/mL).

(c) Synthesis of NH₂-C₅-DPPE

70 mg of DPPE (100 μmol) was suspended in 20 mL of chloroform, 50 μL of TEA and the 1 mL (approx. 200 μmol) solution of Boc-5-amino valeric acid succinimide ester was added, then stirred and reacted overnight at 20° C. After completion of reaction, TEA was extracted using methanol and 3% aqueous citric acid, dehydrated with anhydrous sodium sulfate, and solvents were removed by a rotary evaporator. 1.5 mL of 1 M HCl/acetic acid was then added to the product to solve it and left it stand for one hour at 37° C. After concentrated by a rotary evaporator, it was washed with methanol and chloroform repeatedly, and hydrochloric acid and acetic acid were removed. Then, with silica gel thin layer chromatography for fractionation (#5717, Merck), the resultant product was purified with chloroform/methanol=7/3 mixed solvent as developing solvent. The yield was about 60%.

2. Synthesis of Bromoacetyl (BrAc)-NH-C₅-DPPE

140 mg (1 mmol) of bromoacetic acid was solved in. 30 mL of chloroform, 140 mg of HSI (1.2 mmol) and 250 mg (1.2 mmol) of DCCD were added, and after three hours of reaction at room temperature the solvent was removed by a rotary evaporator and 30 mL of ethyl acetate was added. The resultant white precipitate was filtered, and after the solvent was removed again the precipitate was solved in 10 mL of chloroform.

Approximately 10 mL (50 μmol) of NH₂—C₅-DPPE in chloroform prepared in the step 1, 1 mL of said solution and 50 μl of TEA were added and reacted overnight at room temperature. After completion of the reaction, the solvent was concentrated and the resultant product was purified by means of thin layer chromatography for fractionation with chloroform/methanol=7/3 mixed solvent as developing solvent. The yield was 50%. The final product was diluted with chloroform to the concentration of 1 mM.

3. Preparation of Liposome Reagent

All lipids and aliphatic amines used were solved in chloroform or mixed solvent of chloroform/methanol (2/1). 200 μL of 5 mM DPPC, 100 μL of 10 mM cholesterol, 50 μL of 1 mM BrAc—NH—C₅-DPPE prepared in the step 2 and 25 μL of 5 mM stearyl amine were added to a 10 mL pear-shaped flask and 2 mL of chloroform was further added and mixed vigorously. The solvent was then removed by a rotary evaporator in a water bath at approx. 40° C. 2 mL of chloroform was added again with vigorous stirring and the solvent was removed again by a rotary evaporator. Repeating this procedure several times formed lipid thin layers on a wall of the flask. The flask was then transferred in a desiccator and the solvent was completely removed by approximately one hour of aspiration using a vacuum pump. 100 μL of 1 mg/mL glucose oxidase (hereinafter abbreviated as GOD (Sigma), in 100 mM phosphate buffer pH 7.4 (with 0.85% NaCl, abbreviated as PBS)) was added, the flask was purged with nitrogen and sealed to immerse in a water bath at about 60° C. in about one minute. Subsequently, a vortex mixer was used to shake the flask vigorously until the lipid thin layers on the wall of the flask had disappeared completely. This procedure gave multilayer liposome suspension. After adding small amount of buffer into the liposome suspension, the entire suspension was transferred to a centrifuge tube and a centrifugation procedure at 15,000 rpm and 4° C. for 20 minutes was repeated several times. Finally, the liposome was transferred to a serum tube (Corning) with 10 mM borate buffer (pH 9.0, with 0.85% NaCl; hereinafter referred to as BBS), supernatant obtained after centrifuging once was removed and stored in a refrigerator until use for anti human AFP antibody immobilization reaction described below.

4. Modification of Anti-Human AFP Antibody

Anti human AFP monoclonal antibody was self-prepared by immunizing mice with purified human AFP (Dako) (subclass; IgG₁). 100 μg of this antibody (1 mg/mL; in 0.1M acetate buffer (pH 4.5) was added to 1 mL of pepsin (Sigma) and reacted for one hour at 37° C. Only F(ab′)₂ fraction was then fractionated by high performance liquid chromatography. 10 mg of mercaptoethylamine/HCl was added to the F(ab′)₂ fraction [in 0.1M phosphate buffer (pH 6.0)] and reacted at 37° C. for 90 minutes, and protein fraction containing free SH groups (Fab′) was fractionated by gel filtration (Sephadex G −25, BBS). For the solution of this protein fraction, OD 280 nm was 1.

5. Immobilization of Anti-Human AFP Antibody (Fab′) to Liposome

The liposome suspension and a solution of Fab′ were mixed, and stirred and reacted at 20° C. for 44 hours. After completion of the reaction, this was washed three times with gelatin-veronal buffer (hereinafter referred to as GVB⁻). Finally, the resultant liposome reagent was suspended in 2 mL of GVB− and stored at 4° C.

6. Immobilization of Rabbit Anti-Human AFP Antibody (Polyclonal; DAKO) to Magnetic Particles

Rabbit anti-human AFP antibody was treated by cross-linking agent, SPDP (Pharmacia). The treated antibody was reacted with biotin (Sigma) provided by similar treatment and reduced by dithiothreitol (Sigma) to produce biotinylated rabbit anti-human AFP antibody. The labeled antibody was reacted with avidin immobilized-magnetic particles (Chisso) to prepare solution of magnetic particles immobilized with rabbit anti-human AFP antibody.

7. Plotting a Standard Curve for Measurement of Human AFP

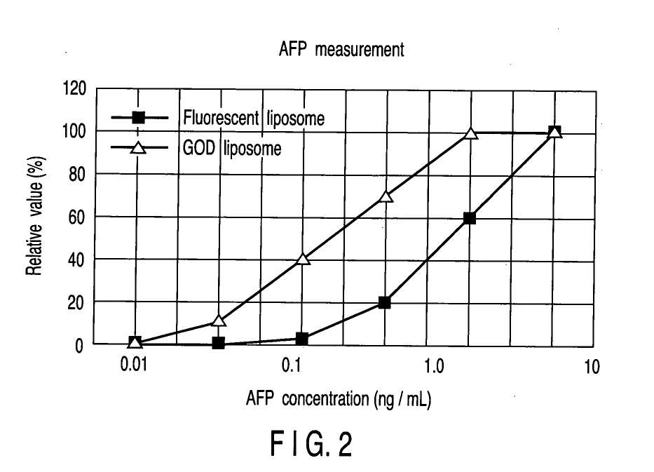

Standard solutions of human AFP (0.01-1000 ng/mL) (Dako) were prepared with GVB²⁺ (prepared by adding 0.5 mM MgCl₂ and 0.15 mM CaCl₂ to GVB⁻. To each well of a microtiter plate, 100 μl of these solutions and ×10 diluted solution of 100 μL of the magnetic particles solution were added and incubated at 37° C. for 5 minutes and cooled to 30° C. This resulted in the aggregated magnetic particles and collection with a magnet could be achieved. The particles collected with a magnet were washed twice with 100 μL of GVB²⁺. After washing, 100 μL of ×10 diluted solution of the liposome reagent described above was added, and subjected for reaction at 37° C. for five minutes. After completion of the reaction, the temperature was cooled down to 30° C. to aggregate and collect the magnetic particles. After washing twice as described above, 100 μL of pure water was added, and after one minute at 37° C., 100 μL of 1% glucose (PBS solution) was added, and enzymatic reaction was monitored using a custom-made micro working electrode (To a DKK). The value of current after insertion of the electrode was recorded. Relative value of current was calculated as follows.
Relative value of current=(Ae−Ao)/(Am−Ao)×100 (%)
wherein Ae: value of current actually measured for respective concentrations of human AFP, Ao: value of current generated when equal amount of GVB²⁺ was added in place of human AFP solution, Am: value of current at concentration generating maximum value of current. For comparison, we also evaluated a system wherein liposome reagent encapsulating 0.1 M carboxy fluorescein (fluorescent substance) in stead of enzyme GOD was used. In this case, measurement was performed using a spectrofluorometer for a microtiter plate (MTP-32, Corona Electrics) with excitation wavelength of 460 nm and emission wavelength of 505 nm (custom-made filter). Relative fluorescence intensity was calculated according to the following equation.
   Relative fluorescence intensity=(Fₑ−Fₒ)/(Fₘ−F₀)×100 (%)
wherein, Fₑ: fluorescence intensity actually measured for respective concentrations of human AFP, F₀: fluorescence intensity when equal amount of GVB²⁺ was added in place of human AFP solution, Fₘ: maximum fluorescence intensity within this concentration range. The results of these measurements are shown in FIG. 2. As shown in this figure, it was demonstrated electrochemical measurement is approximately two orders more sensitive than fluorescent measurement. In addition, it was also demonstrated we could measure AFP concentration of an actual serum sample using the standard curves.

### Example 2

### Measurement of Human AFP Using Polymer Microcapsule Reagent Immobilized with Anti-Human AFP Monoclonal Antibodies

Microcapsules were prepared with pH responsive polymers in stead of liposome reagents described above (reagent 1). The encapsulated substance was GOD. Chemical adsorption method was used for antibody immobilization. Other conditions were same as mentioned above for the liposome reagent system (same as Example 1), except BBS of pH 5 was used in stead of water to disrupt the microcapsules. This experiment revealed that measurement sensitivity was achieved similar to that obtained by the liposome reagent of Example 1.

### Example 3

### Qualitative Ultratrace Detection of Trinitrotoluene (TNT) Using a Liposome Reagent Immobilized with Anti-TNT Monoclonal Antibody

Anti-TNT monoclonal antibody and rabbit anti-DNP (dinitrophenyl) antibody (immunological analysis reagent 4; second antibody) were self-prepared according to a method of producing antibody against hapten (see “Tanpakusitu Kakusan Koso”, extra ed., issue of December, 1996, p. 84-87). The liposome reagent and magnetic particles were obtained in a manner similar to Example 1. 0.1-1,000 pg/mL standard solutions of TNT (GVB²⁺ was used) were prepared and measured in a similar manner as Example 1. 10% increase in relative value of current was found at 0.1 pg/mL of TNT as early as after five minutes, demonstrating qualitative determination was possible. When we assume 1 L of sampled gas is solved in 1 mL of GVB²⁺, measurement within this concentration range is equivalent to TNT measurement at ppt level. Thus, it seems possible to examine explosives at an airport.

### Example 4

### Example When Porous Carriers Carrying Redox Enzyme is Used as an Immunological Analysis Carrier

It is possible to perform similar experiments in Example 1 using porous carriers (porous microparticles) in place of liposome. Sepharose CL (Amersham) is used as the porous microparticles. AFP polyclonal antibodies and GOD applied for Example 1 were mixed at the weight ratio of 1:10 and immobilized to the surface of the microparticles by chemical biding method. AFP standard solutions can be measured with similar procedures as Example 1, but the step for disrupting microparticles after collection by a magnet will be omitted. Enzymatic activity was measured by adding substrate solution (glucose) directly.

It is expected that similar results as Example 1 will be obtained, but detection ability of this method will be one order less sensitive.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general invention concept as defined by the appended claims and their equivalent.

1. An immunological analysis carrier comprising: at least one of an antibody against a target substance, an agent capable of specifically binding a target substance, a part of an antibody against a target substance and a part of an agent capable of specifically binding a target substance, the at least one of the antibody, the agent, the part of the antibody and the part of the agent being immobilized on the carrier's surface; and a redox enzyme capable of generating an electrochemically active substance carried on the carrier's surface or encapsulated in the carrier.

2. An immunological analysis carrier according to claim 1, wherein the carrier is in the form of a microcapsule encapsulating a redox enzyme, the redox enzyme being capable of generating an electrochemically active substance.

3. An immunological analysis carrier according to claim 1, wherein immobilization is achieved using a spacer.

4. An immunological analysis carrier according to claim 2, wherein immobilization is achieved using a spacer.

5. An immunological analysis carrier according to claim 1, wherein the redox enzyme capable of generating an electrochemically active substance is glucose oxidase.

6. An immunological analysis carrier according to claim 2, wherein the redox enzyme capable of generating an electrochemically active substance is glucose oxidase.

7. A kit for immunological analysis comprising: an immunological analysis carrier according to claim 1; a separating particle having an antibody against a target substance or a binding agent capable of specifically biding the target substance or a part thereof immobilized on its surface.

8. A kit according to claim 7, wherein the immunological analysis carrier is in the form of a microcapsule encapsulating a redox enzyme, the redox enzyme being capable of generating an electrochemically active substance.

9. A kit according to claim 7, further comprising a substrate for the enzyme.

10. A kit according to claim 8, further comprising a substrate for the enzyme.

11. A kit according to claim 7, wherein immobilization is achieved using a spacer.

12. A kit according to claim 8, wherein immobilization is achieved using a spacer.

13. A kit according to claim 7, wherein the redox enzyme capable of generating an electrochemically active substance is glucose oxidase.

14. A kit according to claim 8, wherein the redox enzyme capable of generating an electrochemically active substance is glucose oxidase and the substrate for the enzyme is glucose.

15. A method of immunological analysis comprisising;
mixing the separating particle according to claim 7 with target substance solution to couple an antibody or a binding agent immobilized to the separating particle with the target substance,
mixing the separating particle with the immunological analysis carrier according to claim 1 to couple the target substance with the immunological analysis carrier,
separating the separating particle from the target substance solution,
adding a substrate for the enzyme into the mixture,
reacting the redox enzyme and the substrate for the enzyme by disrupting the immunological analysis carrier if the redox enzyme is encapsulated in the carrier, and
measuring a reaction between the redox enzyme and the substrate.

16. A method according to claim 15, wherein the reaction between the enzyme and the substrate is measured electrochemically.

17. A method according to claim 15, wherein the immunological analysis carrier is in the form of a microcapsule encapsulating a redox enzyme capable of generating an electrochemically active substance.

18. A method according to claim 16, wherein the immunological analysis carrier is in the form of a microcapsule encapsulating a redox enzyme capable of generating an electrochemically active substance.

19. A method according to claim 17, wherein the redox enzyme capable of generating the electrochemically active substance is glucose oxidase.

20. A method according to claim 18, wherein the redox enzyme capable of generating an electrochemically active substance is glucose oxidase.
